(19) United States
(12) Patent Application Publication
(10) Pub. No.: US 2026/0045328 A1
(43) Pub. Date: Feb. 12, 2026

(54) COMPUTER SYSTEM, INFORMATION PROCESSING METHOD, AND NON-TRANSITORY COMPUTER-READABLE STORAGE MEDIUM

(71) Applicant: Hitachi, Ltd., Tokyo (JP)

(72) Inventors: Ryo IKEGAMI, Tokyo (JP); Kunihiko KIDO, Tokyo (JP); Yoichi NAKAMOTO, Tokyo (JP); Ryota NOMA, Tokyo (JP)

(21) Appl. No.: 19/232,850

(22) Filed: Jun. 9, 2025

(30) Foreign Application Priority Data
Aug. 7, 2024 (JP) 2024-130784

(51) Int. Cl. G16H 10/20

(52) U.S. Cl. CPC G16H 10/20

(57) ABSTRACT

A computer system is coupled for access to a first database that stores clinical data including, as an item, information acquired on a clinical site and relating to a patient provided with a therapy and a second database that stores non-clinical data including, as an item, information acquired for an investigation purpose and relating to a patient group provided with a therapy. The computer system selects a predetermined number of pieces of the clinical data from the first database, and aggregate the selected predetermined number of pieces of the clinical data, to thereby generate comparative data; and executes, through use of the non-clinical data and the comparative data which are the same in therapy content, statistical analysis for analyzing a difference in the item.

### CLAIM OF PRIORITY

The present application claims priority from Japanese patent application JP 2024-130784 filed on Aug. 7, 2024, the content of which is hereby incorporated by reference into this application.

### BACKGROUND OF THE INVENTION

This invention relates to a technology for supporting efficient use of real-world data.

In recent years, in the field of medical drug development, use of not only data on specific patients collected through clinical trials and clinical research, but also data collected at actual clinical sites is attracting attention. Information based on medical treatment acquired at the actual clinical sites is referred to as “real-world data (RWD),” and investigation relating to therapy situations, therapy efficacy, and the like is executed for various diseases.

In JP 2022-180080 A, there is disclosed a technology involving structuring text information such as electronic medical record data being one type of RWD and identifying, through use of the structured information and information on a reference of a clinical trial, a patient being a candidate for a clinical trial target.

### SUMMARY OF THE INVENTION

Hitherto, therapy situations and drug efficacy have been grasped for limited patients as targets such as those in the clinical trial and the clinical study. Individualization is in progress in the current medical field, and hence it is difficult to grasp, from the data on only some of the patients, the therapy situations and the drug efficacy at the actual clinical sites. Thus, the use of the RWD enabling analysis targeting all patients is in progress.

A degree of difficulty of the use of the RWD varies depending on types of diseases, and increases as the therapy pattern varies more. An issue that may obstruction of the use of the RWD is that an enormous amount of temporal/human costs is required for analysis because the number of pieces of data is enormous and the pieces of data are unstructured unlike the data on the clinical trials and the clinical study.

This invention has an object to support efficient use of RWD.

A representative example of the present invention disclosed in this specification is as follows: a computer system, comprises a processor; a storage device coupled to the processor; and a communication interface coupled to the processor. The computer system is coupled for access to a first database that stores clinical data including, as an item, information acquired on a clinical site and relating to a patient provided with a therapy and a second database that stores non-clinical data including, as an item, information acquired for an investigation purpose and relating to a patient group provided with a therapy. The processor is configured to: select a predetermined number of pieces of the clinical data from the first database, and aggregate the selected predetermined number of pieces of the clinical data, to thereby generate comparative data; and execute, through use of the non-clinical data and the comparative data which are the same in therapy content, first statistical analysis for analyzing a difference in the item, and record a result of the first statistical analysis.

According to the at least one embodiment of this invention, it is possible to present information for extracting clinical data (RWD) to be analyzed. As a result, the RWD can efficiently be used. Problems, configurations, and effects other than those described above become apparent from the following description of at least one embodiment.

### BRIEF DESCRIPTION OF THE DRAWINGS

The present invention can be appreciated by the description which follows in conjunction with the following figures, wherein:

FIG. 1 is a diagram for illustrating an example of the configuration of the system in a first embodiment of this invention;

FIG. 2 is a diagram for illustrating an example of a hardware configuration of a therapy situation/drug efficacy investigation system in the first embodiment;

FIG. 3 is a table for showing an example of information stored in a regimen management DB in the first embodiment;

FIG. 4 is a table for showing an example of information stored in a non-clinical data DB in the first embodiment;

FIG. 5 is a table for showing an example of information stored in a clinical data DB in the first embodiment;

FIG. 6 is a table for showing an example of a statistical analysis method DB in the first embodiment;

FIG. 7 is a table for showing an example of an analysis result DB in the first embodiment;

FIG. 8A and FIG. 8B are tables for showing examples of information stored in an extracted information DB in the first embodiment;

FIG. 9 is a flowchart for illustrating overview of the processing executed by the therapy situation/drug efficacy investigation system in the first embodiment;

FIG. 10 is a flowchart for illustrating an example of pre-statistical analysis processing executed by the therapy situation/drug efficacy investigation system in the first embodiment; and

FIG. 11 is a flowchart for illustrating an example of statistical analysis processing executed by the therapy situation/drug efficacy investigation system in the first embodiment.

### DETAILED DESCRIPTION OF THE PREFERRED EMBODIMENTS

Hereinafter, an embodiment of the present invention will be described with reference to the drawings. The embodiment is an example for explaining the present invention, and appropriate omissions and simplifications are made for clarity of explanation. The present invention can be implemented in various other forms. Unless otherwise specified, each component may be singular or plural.

The position, size, shape, range, and the like of each configuration illustrated in the drawings and the like may not represent the actual position, size, shape, range, and the like in order to facilitate understanding of the invention. Therefore, the present invention is not limited to the position, size, shape, range, and the like disclosed in the drawings and the like.

As an example of various types of information, expressions such as “table,” “list,” and “queue” are used for description, but various types of information may be expressed in a data structure other than those structures. For example, various types of information such as “xx table,” “xx list,” and “xx queue” may be expressed in “xx information.” When identification information is described, expressions such as “identification information,” “identifier,” “name,” “ID,” and “number” are used, and those expressions are exchangeable with one another.

When there exist a plurality of components having the same function or similar functions, those components are sometimes described by adding different suffixes to the same reference numeral. Moreover, when it is not required to distinguish those plurality of components from one another, those components are sometimes described while omitting the suffixes.

In at least one embodiment of this invention, processing executed through execution of a program is described in some cases. Here, a computer uses a processor (for example, a CPU or a GPU) to execute the program to execute processing defined by the program while using a storage resource (for example, a memory), an interface device (for example, a communication port), and the like. Thus, a subject of the processing executed through execution of the program may be the processor. Similarly, the subject of the processing executed through the execution of the program may be a controller, a device, a system, a computer, or a node including the processor. It is only required that the subject of the processing executed through the execution of the program be an arithmetic unit, and may include a dedicated circuit which executes specific processing. Here, the dedicated circuit is, for example, a field-programmable gate array (FPGA), an application-specific integrated circuit (ASIC), and a complex programmable logic device (CPLD).

The program may be installed on the computer from a program source. The program source may be, for example, a program distribution server or a computer-readable storage medium. When the program source is the program distribution server, the program distribution server includes a processor and a storage resource which stores the program to be distributed, and the processor of the program distribution server may distribute the program to be distributed to another computer. Moreover, in the at least one embodiment, two or more programs may be implemented as one program, and one program may be implemented as two or more programs

### First Embodiment

In the at least one embodiment, as an example of an information system which supports therapy situation/drug efficacy investigation in actual clinical sites through use of RWD, description is given of a technology which extracts a patient group to be analyzed from the RWD. With this information system, for example, it is possible to suppress an analysis cost for the drug efficacy and the therapy situations in the actual clinical sites, to thereby make contribution in terms of economy.

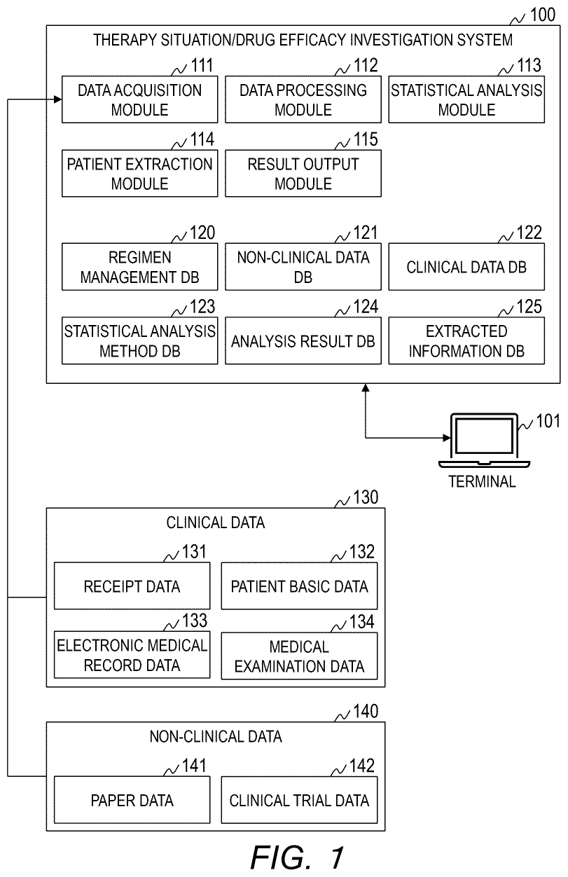

First, description is given of a configuration example of the system with reference to FIG. 1. FIG. 1 is a diagram for illustrating an example of the configuration of the system in a first embodiment of this invention.

The system includes a therapy situation/drug efficacy investigation system (information system) 100 and a terminal 101.

The therapy situation/drug efficacy investigation system 100 is a system which executes reading of data, processing of the data, statistical analysis, extraction of a patient group, and output of an analysis result. The therapy situation/drug efficacy investigation system 100 includes a data acquisition module 111, a data processing module 112, a statistical analysis module 113, a patient extraction module 114, and a result output module 115.

The data acquisition module 111 acquires clinical data 130 and non-clinical data 140.

The clinical data 130 is data corresponding to RWD data acquired at clinical sites, and includes, for example, receipt data 131, patient basic data 132, electronic medical record data 133, and medical examination data 134. Herein, one piece of clinical data 130 exists for one patient. The data acquisition module 111 acquires the clinical data 130 on a plurality of patients from the clinical sites.

The receipt data 131 is data including information on regimens obtained by extracting drug information on the patients and the like. The regimen refers to a combination of therapies, and means a therapy plan obtained by combining a plurality of drugs, for example, a cancerous region and the like. The patient basic data 132 is data including information characterizing the individual patient such as an ID, the age, the gender, the place of residence, and a disease name of the patient. The electronic medical record data 133 is data including daily medical treatment records and therapy records. The medical examination data 134 is data including results of various medical examinations executed for the patient such as a result of a clinical examination and image data.

The non-clinical data 140 is data collected for the purpose of the investigation, and includes, for example, paper data 141 and clinical trial data 142.

The paper data 141 and the clinical trial data 142 are data obtained by extracting, from published papers and research results, background information on patients, therapy content, therapy results, and the like. In the first embodiment, it is assumed that one piece of non-clinical data 140 exists for one patient group. It is possible to acquire one or more pieces of non-clinical data 140 from the papers and the research results. The data on one patient in the papers and the research results can be treated as one piece of non-clinical data 140.

The data processing module 112 processes the clinical data 130 and the non-clinical data 140 acquired by the data acquisition module 111 into a data form to which the statistical analysis can be applied. The acquired data and the processed data are stored in a non-clinical data DB 121 and a clinical data DB 122.

The statistical analysis module 113 executes statistical analysis applied to the clinical data 130 and the non-clinical data 140 processed by the data processing module 112. A result of the statistical analysis is stored in an analysis result DB 124.

The patient extraction module 114 extracts the clinical data 130 satisfying a predetermined extraction condition based on the result of the statistical analysis.

The result output module 115 outputs the clinical data 130 extracted by the patient extraction module 114 and the like.

It is assumed that each function of the therapy situation/drug efficacy investigation system 100 can be controlled from the terminal 101. Details of each DB held by the therapy situation/drug efficacy investigation system 100 are described later.

The terminal 101 is a terminal to be operated by a user who uses the therapy situation/drug efficacy investigation system 100, and is, for example, a general-purpose computer, a smartphone, or a tablet terminal.

The therapy situation/drug efficacy investigation system 100 and the terminal 101 are wirelessly or wiredly coupled to each other. Moreover, a plurality of terminals 101 may be coupled to the therapy situation/drug efficacy investigation system 100.

As communication between the therapy situation/drug efficacy investigation system 100 and the terminal 101, for example, the 5th generation mobile communication system, so called 5th generation (5G), which has achieved “massive machine type communications” and “ultra-low latency,” can be used. Through active use of the characteristics of a new system of 5G or later, even when a large number of terminals 101 are simultaneously coupled, communication delay can be suppressed.

The therapy situation/drug efficacy investigation system 100 may be implemented through use of a cloud system. Moreover, for the wireless communication between the therapy situation/drug efficacy investigation system 100 on the cloud system and the terminal 101, a new system of 5G or later may be used.

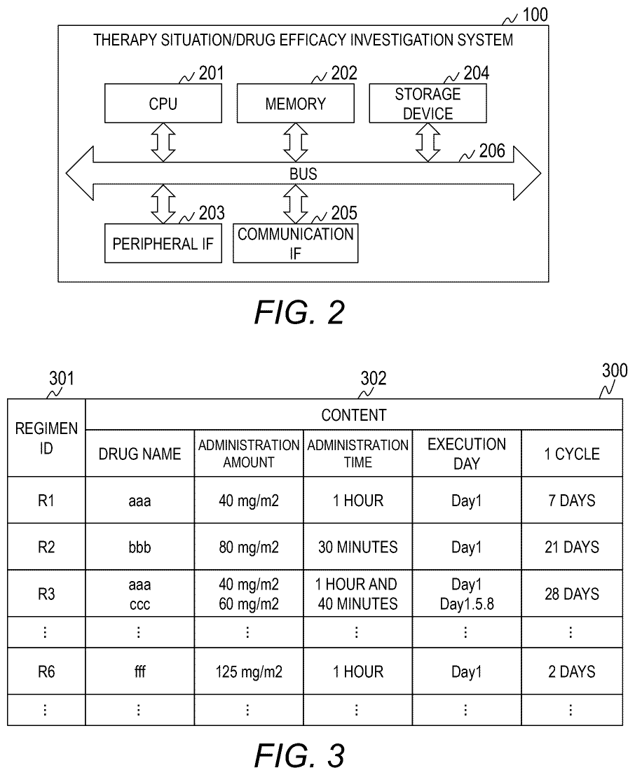

FIG. 2 is a diagram for illustrating an example of a hardware configuration of the therapy situation/drug efficacy investigation system 100 in the first embodiment.

The therapy situation/drug efficacy investigation system 100 includes a central processing unit (CPU) 201, a memory 202, a peripheral IF 203, a storage device 204, and a communication IF 205. The hardware components are coupled to one another for communication via a bus 206.

The CPU 201 is a calculation device which executes a program stored in the storage device 204. The memory 202 is a volatile storage device and stores the program executed by the CPU 201. Moreover, the memory 202 may be used as a working area and a temporary buffer.

The storage device 204 is formed of a magnetic disk device, a flash read-only memory (ROM), and the like, and stores an OS, various drivers, various application programs, and various types of information used by the programs.

The CPU 201 reads out the program stored in the storage device 204, loads the program onto the memory 202, and executes the program, resulting in implementation of the function modules being the data acquisition module 111, the data processing module 112, the statistical analysis module 113, the patient extraction module 114, and the result output module 115.

The peripheral IF 203 is an interface for coupling to various peripheral devices such as input/output devices, for example, a mouse, a keyboard, and a monitor, and an external storage, for example, a universal serial bus (USB) memory.

The communication IF 205 is an interface for the therapy situation/drug efficacy investigation system 100 to communicate to and from the outside. The number of communication IFs 205 may be two or more.

FIG. 3 is a table for showing an example of information stored in a regimen management DB 120 in the first embodiment.

The regimen management DB 120 stores a table 300 as shown in FIG. 3 for each disease. The table 300 shown in FIG. 3 is a table for managing regimens in therapy for “cancer,” and stores records each including a regimen ID 301 and content 302. One record exists for one regimen.

The regimen ID 301 is a field which stores an ID indicating identification information on the regimen.

The content 302 is a field group which stores specific content of the regimen. For example, the content 302 includes fields such as a drug name, an administration amount, an administration time, an execution day, and one cycle. The above-mentioned fields are examples, and the fields are not limited to this example.

For example, “drug name” of a record having “R1” in the regimen ID 301 indicates that a used drug name is “aaa.” A case in which the same drug is not used in a plurality of regimens is shown in FIG. 3, but regimens having the same drug but different in information such as the administration amount are managed as different regimens.

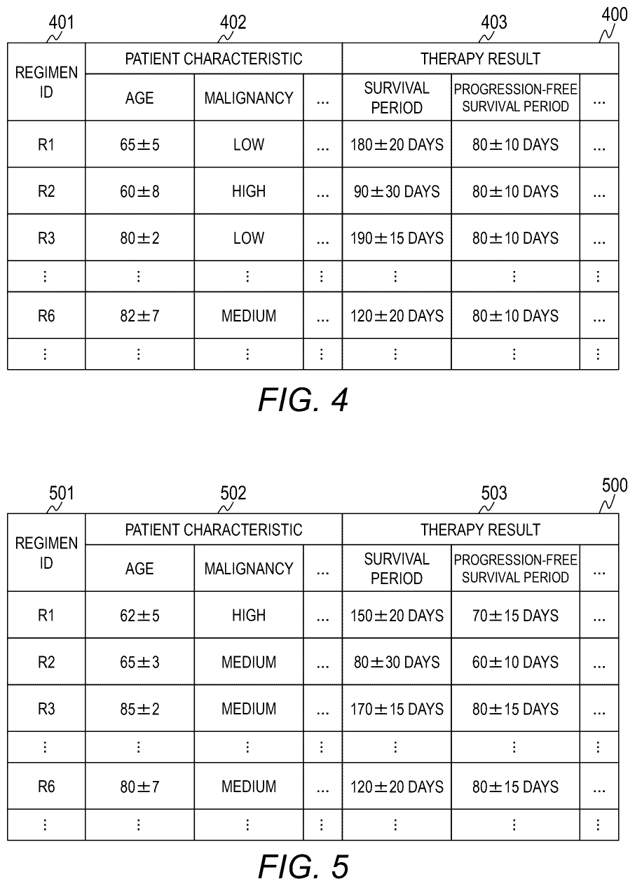

FIG. 4 is a table for showing an example of information stored in the non-clinical data DB 121 in the first embodiment.

The non-clinical data DB 121 stores the acquired non-clinical data 140 and stores a table 400 as shown in FIG. 4 for each disease. The table 400 shown in FIG. 4 is a table relating to a patient group of “cancer,” and stores records each including a regimen ID 401, a patient characteristic 402, and a therapy result 403. One record exists for one regimen.

The regimen ID 401 is the same field as the regimen ID 301.

The patient characteristic 402 is a field group which stores information relating to characteristics of patients forming the patient group. The patient characteristic 402 includes information unique to the patients such as the gender and the age and information indicating a disease state of the patient group such as a disease name and malignancy of the disease.

The therapy result 403 is a field group which stores information relating to a therapy result. The therapy result 403 includes a survival period and a progression-free survival period.

For example, a patient group (record) having “1” in the regimen ID 401 has an average of 65±5 in age, have “low” in the malignancy of the disease, have “180±20 days” in the survival period, and have “80±10 days” in the progression-free survival period.

The patient characteristic 402 and the therapy result 403 are used in the statistical analysis.

In the following description, the characteristic, the disease state, the therapy result, and the like of the patients included in the clinical data 130 and the non-clinical data 140 are collectively referred to as “items.”

FIG. 5 is a table for showing an example of information stored in the clinical data DB 122 in the first embodiment.

The clinical data DB 122 stores the acquired clinical data 130 and stores a table 500 as shown in FIG. 5 for each disease. The table 500 shown in FIG. 5 is a table relating to a patient of “cancer,” and stores records each including a regimen ID 501, a patient characteristic 502, and a therapy result 503. One record is data for comparison used in the statistical analysis which makes comparison with the non-clinical data 140. One record exists for one regimen.

The regimen ID 501 is the same field as the regimen ID 301. The patient characteristic 502 is the same field group as the patient characteristic 402. The therapy result 503 is the same field group as the therapy result 403.

For example, a patient group (record) having “1” in the regimen ID 501 has an average of 62±5 in age, have “high” in the malignancy of the disease, have “150±20 days” in the survival period, and have “70±15 days” in the progression-free survival period.

The patient characteristic 502 and the therapy result 503 are used in the statistical analysis.

As required, the receipt data 131, the patient basic data 132, the electronic medical record data 133, and the medical examination data 134 may be selected or combined to generate data to be stored in the table 500.

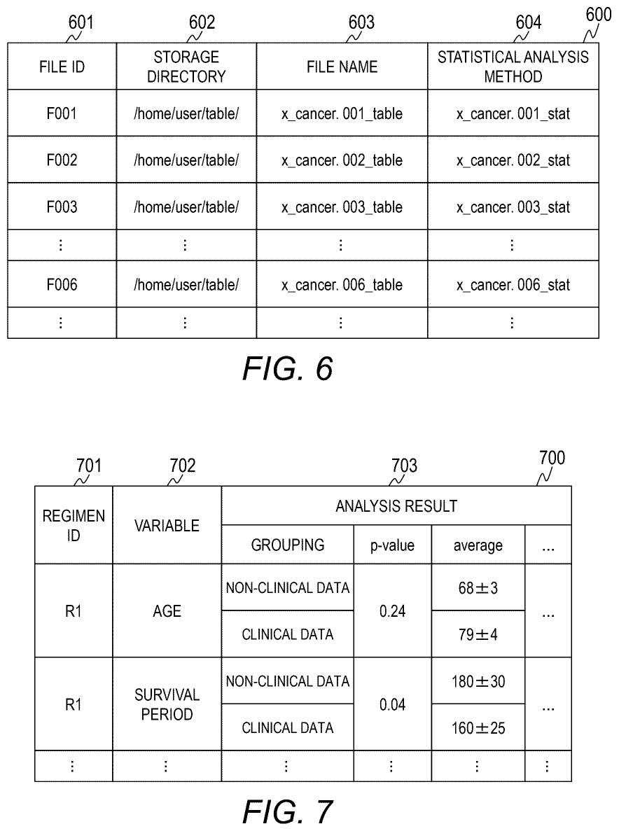

FIG. 6 is a table for showing an example of a statistical analysis method DB 123 in the first embodiment.

The statistical analysis method DB 123 stores a table 600. The table 600 stores records each including a file ID 601, a storage directory 602, a file name 603, and a statistical analysis method 604. One record exists for one result of the statistical analysis.

The file ID 601 is a field which stores an ID identifying a file in which the analysis result is to be stored. The storage directory 602 is a field which stores a directory name which stores the file. The file name 603 is a field which stores a name of the file. The statistical analysis method 604 is a field which stores a program used for the statistical analysis.

For example, the analysis result having “F001” in the file ID 601 is stored in a directory “/home/user/table/,” and indicates that the file name is “x_cancer.001_table.” Moreover, it is indicated that the analysis result is obtained through the statistical analysis which uses a program “x_cancer.001_stat.”

FIG. 7 is a table for showing an example of the analysis result DB 124 in the first embodiment.

The analysis result DB 124 stores the analysis result in a file format. The analysis result is, for example, a table 700 as shown in FIG. 7. The table 700 stores records each including a regimen ID 701, a variable 702, and an analysis result 703.

The regimen ID 701 is the same field as the regimen ID 301.

The variable 702 is a field which stores a name of an item to be compared between the clinical data 130 and the non-clinical data 140. In the first embodiment, the statistical analysis of comparing a difference in item is executed.

The analysis result 703 is a field group which stores the result of the statistical analysis. In the analysis result 703, indices calculated through the statistical analysis such as an average value, a median, a probability value such as a p-value, a probability distribution, and an area are stored.

For example, a first record indicates a result of the statistical analysis which uses data having “R1” in the regimen ID 701 and has “age” as a variable. The p-value is “0.24” and hence it is found that a significant difference does not exist in terms of the age. Moreover, it is found that the average (“average”) of the ages of the patient group corresponding to the non-clinical data 140 is “68±3,” and the average (“average”) of the ages of the patient group corresponding to the clinical data 130 is “79±4.”

The user uses the terminal 101 to input, as an extraction condition, a conditional expression using a variable and a threshold value for an index, to thereby be able to retrieve and refer to a regimen satisfying the extraction condition from the analysis result. For example, it is possible to input, as the extraction condition, “the p-value of the age is 0.05 or less, and the p-value of the survival period is 0.05 or less.”

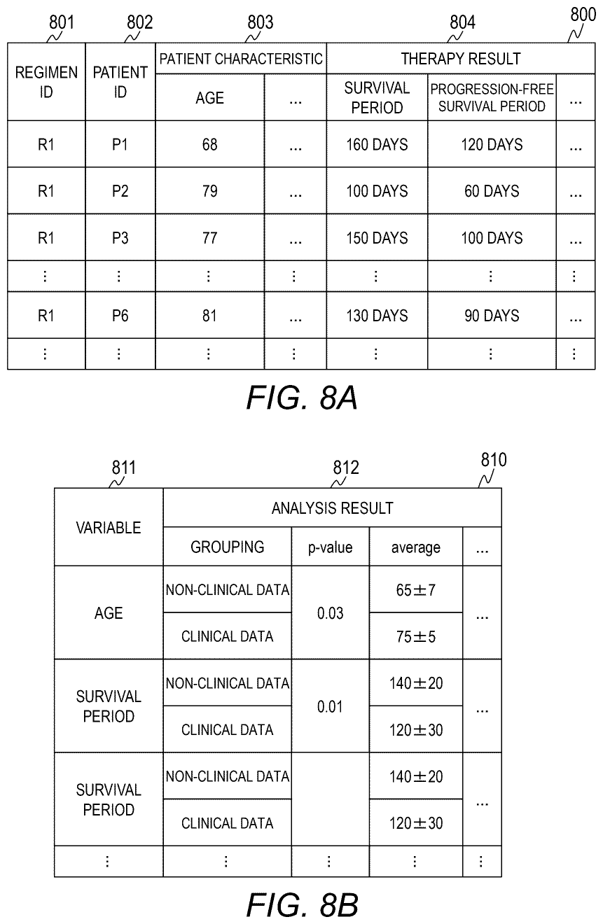

FIG. 8A and FIG. 8B are tables for showing examples of information stored in an extracted information DB 125 in the first embodiment.

In the extracted information DB 125, a table 800 and a table 810 are stored. The table 800 is a table which stores the clinical data 130 on the regimens satisfying the extraction condition. The table 810 is a table which stores the result of the statistical analysis through use of the clinical data 130 on the regimens satisfying the extraction condition and the non-clinical data 140.

The table 800 stores records each including a regimen ID 801, a patient ID 802, a patient characteristic 803, and a therapy result 804. One record exists for one patient.

The regimen ID 801 is the same field as the regimen ID 301. The patient ID 802 is a field which stores an ID identifying a patient managed in the clinical data 130. The patient characteristic 803 is a field group which stores information relating to characteristics of a patient. The therapy result 804 is a field group which stores information relating to a therapy result.

For example, in a first record, information relating to the patient characteristic and the therapy result of a patient having “P1” in the patient ID 802 is stored. Specifically, the first record indicates that the age of the patient is “68,” the survival period is “160 days,” and the progression-free survival is “120 days.”

The table 810 stores records each including a variable 811 and an analysis result 812. There exist as many entries as the number of analyzed variables. The variable 811 and the analysis result 812 are the same fields as the variable 702 and the analysis result 703, respectively.

The user can operate the terminal 101 to appropriately select the variables to be acquired. For example, the user can select “age,” “gender,” “malignancy,” and “survival period.”

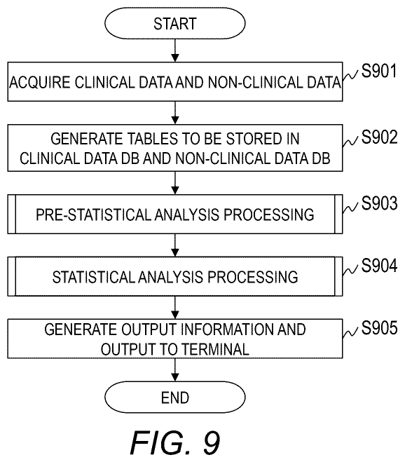

Description is now given of processing executed by the therapy situation/drug efficacy investigation system 100. FIG. 9 is a flowchart for illustrating overview of the processing executed by the therapy situation/drug efficacy investigation system 100 in the first embodiment.

The data acquisition module 111 of the therapy situation/drug efficacy investigation system 100 acquires the clinical data 130 and the non-clinical data 140 (S901).

Next, the data processing module 112 of the therapy situation/drug efficacy investigation system 100 uses the clinical data 130 and the non-clinical data 140 to generate the tables 400 and 500 (S902). Description is now given of generation methods for the tables 400 and 500.

The data processing module 112 processes the non-clinical data 140 in accordance with the data structure of the table 400. When the regimen ID is not included in the non-clinical data 140, the data processing module 112 identifies the regimen based on the information included in the non-clinical data 140 and the regimen management DB 120, and adds the regimen ID to the non-clinical data 140. The data processing module 112 aggregates, for each disease, the processed non-clinical data 140, to thereby generate the table 400. When the data structure of the non-clinical data 140 is the same as the data structure of the table 400, the data processing module 112 can generate the table 400 without processing the non-clinical data 140.

The data processing module 112 generates, for each disease, a set of pieces of clinical data 130 serving as a population, and samples representative clinical data 130 from the population. The data processing module 112 processes the sampled clinical data 130 in accordance with the data structure of the table 500. When the regimen ID is not included in the clinical data 130, the data processing module 112 identifies the regimen based on the information included in the clinical data 130 and the regimen management DB 120, and adds the regimen ID to the clinical data 130. The data processing module 112 applies, for each disease, the statistical processing to the processed clinical data 130 having the same regimen ID, to thereby generate the record of the table 500. The data processing module 112 aggregates the records for each disease, to thereby generate the table 500.

The condition for the sampling may manually be set through use of the terminal 101, or may be set in the program implementing the data processing module 112. For example, the characteristics of the patients such as the age and the gender may manually be set as the condition for the sampling, or an algorithm of clustering the patient group for each regimen and selecting a representative patient from the cluster may be set in the data processing module 112.

Next, the therapy situation/drug efficacy investigation system 100 uses the tables 400 and 500 to execute pre-statistical analysis processing (S903). Details of the pre-statistical analysis processing are described later.

Next, the therapy situation/drug efficacy investigation system 100 extracts, from the clinical data DB 122, the clinical data 130 on the patients satisfying the extraction condition, and uses the non-clinical data 140 and the extracted clinical data 130 to execute the statistical analysis processing (S904). Details of the statistical analysis processing are described later.

Next, the therapy situation/drug efficacy investigation system 100 generates output information based on results of the pre-statistical analysis processing and the statistical analysis processing, and outputs the output information to the terminal 101 (S905). For example, the output information including the tables 700, 800, and 810 is generated.

As a presentation method for the output information, various forms such as a table form, a graph form, and a form of a network diagram can be employed.

The amount of data processed by the sampling is small, and hence the pre-statistical analysis processing can be executed at a low cost.

Detailed statistical analysis can be executed by acquiring the clinical data 130 satisfying the extraction condition based on the result of the pre-statistical analysis processing. For example, when a significant difference in the item exists between the clinical data 130 and the non-clinical data 140, this difference means that the clinical site is different from the research result. Thus, by identifying the item having the significant difference and the regimen, it is possible to identify a patient group to be investigated for grasping new knowledge.

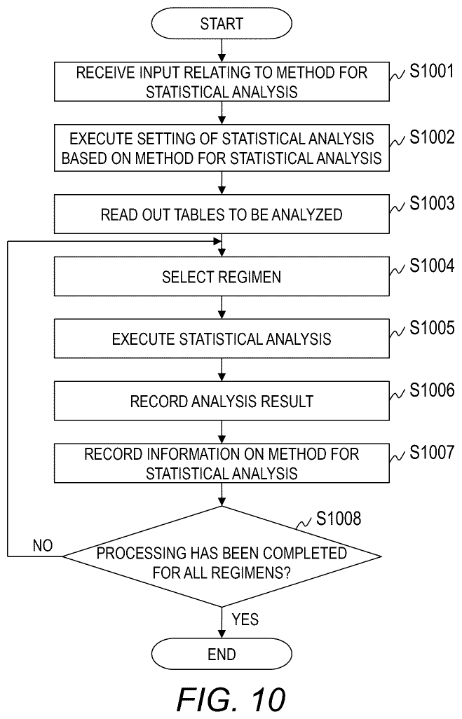

FIG. 10 is a flowchart for illustrating an example of the pre-statistical analysis processing executed by the therapy situation/drug efficacy investigation system 100 in the first embodiment.

The statistical analysis module 113 receives input relating to the method for the statistical analysis (S1001). At this time, input for the type of the disease to be analyzed is also received. The selectable methods for the statistical analysis can be selected, in accordance with the purpose, in a range from general epidemiological statistics and medical statistics to a mathematical program. The method for the statistical analysis in the first embodiment is a method of analyzing whether or not the difference in the item exists between two patient groups.

Next, the statistical analysis module 113 executes setting for executing the statistical analysis based on the method for the statistical analysis (S1002). The setting of the method for the statistical analysis varies in accordance with the method for the statistical analysis to be used.

Next, the statistical analysis module 113 reads out the tables 400 and 500 to be analyzed from the non-clinical data DB 121 and the clinical data DB 122, respectively (S1003). At this time, the statistical analysis module 113 generates a list of the regimen IDs common to the tables 400 and 500 to be analyzed.

Next, the statistical analysis module 113 selects a regimen (S1004).

Next, the statistical analysis module 113 uses records including the selected regimen ID from the tables 400 and 500 based on the setting of the method for the statistical analysis, to thereby execute the statistical analysis (S1005). For example, the statistical analysis is executed based on a publicly-known method for a statistical significant difference test, and indices such as the average value, the median, the probability value such as the p-value, the probability distribution, and the area are calculated.

Next, the statistical analysis module 113 records the analysis result in the analysis result DB 124 (S1006). Specifically, the statistical analysis module 113 adds a record including the analysis result to the analysis result DB 124.

Next, the statistical analysis module 113 records the information on the method for the statistical analysis in the statistical analysis method DB 123 (S1007). Specifically, the statistical analysis module 113 adds, to the statistical analysis method DB 123, a record including a storage destination of the analysis result and the information on the method for the statistical analysis.

Next, the statistical analysis module 113 determines whether or not the processing has been completed for all of the regimens (S1008).

When the processing has not been completed for all of the regimens, the statistical analysis module 113 returns the process to Step S1004, and executes similar processing. When the processing has been completed for all of the regimens, the statistical analysis module 113 finishes the pre-statistical analysis processing.

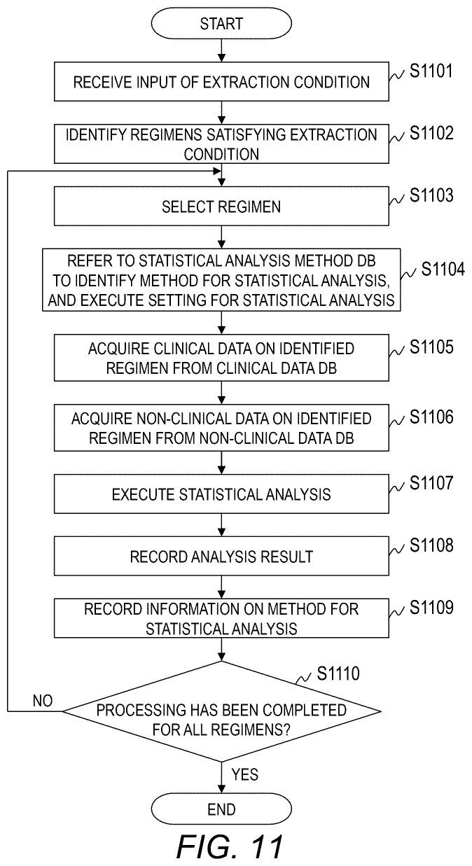

FIG. 11 is a flowchart for illustrating an example of the statistical analysis processing executed by the therapy situation/drug efficacy investigation system 100 in the first embodiment.

The patient extraction module 114 receives input of the extraction condition (S1101). The extraction condition includes at least a pair of condition expressions which use the variable and the threshold value for the index. The threshold value may be freely set, or may be set based on the index calculated in the pre-statistical analysis processing. The type of the disease may be included in the extraction condition.

The patient extraction module 114 refers to, for each disease, the analysis result DB 124 to determine whether or not the extraction condition is satisfied for each regimen, to thereby determine regimens which satisfy the extraction condition (S1102).

The patient extraction module 114 outputs, to the statistical analysis module 113, an execution instruction including information on the specified regimens (list of regimen IDs).

The statistical analysis module 113 selects a regimen (S1103).

The statistical analysis module 113 refers to the statistical analysis method DB 123 based on the file ID of the analysis result corresponding to the identified regimen, to thereby identify the method for the statistical analysis for the selected regimen, and executes the setting (S1104).

The statistical analysis module 113 acquires the clinical data 130 on the identified regimen from the clinical data DB 122 (S1105), and acquires the non-clinical data 140 on the identified regimen from the non-clinical data DB 121 (S1106). Here, all pieces of the clinical data 130 forming the population are acquired.

The statistical analysis module 113 uses the acquired clinical data 130 and the acquired non-clinical data 140 to execute the statistical analysis (S1107). The processing step of Step S1107 is processing similar to the processing step of Step S1005, but the pieces of data to be processed are different from each other.

Next, the statistical analysis module 113 records the analysis result in the extracted information DB 125 (S1108). Specifically, the statistical analysis module 113 adds the extracted clinical data 130 to the table 800 of the extracted information DB 125, and adds the record including the analysis result to the table 810.

Next, the statistical analysis module 113 records the information on the method for the statistical analysis in the statistical analysis method DB 123 (S1109). The processing step of Step S1109 is the same processing as the processing step of Step S1007.

Next, the statistical analysis module 113 determines whether or not the processing has been completed for all of the regimens (S1110).

When the processing has not been completed for all of the regimens, the statistical analysis module 113 returns the process to Step S1103, and executes similar processing. When the processing has been completed for all of the regimens, the statistical analysis module 113 notifies the patient extraction module 114 of the completion of the processing. The patient extraction module 114 finishes the statistical analysis processing after the reception of the notification from the statistical analysis module 113.

According to the at least one embodiment of this invention, the therapy situation/drug efficacy investigation system 100 uses the data on the patient group generated from the sampled clinical data 130 and the non-clinical data 140 to execute the statistical analysis, to thereby be able to generate the information (analysis result) for identifying the analysis target. The user extracts, based on this information, the clinical data 130 satisfying the predetermined extraction condition, and can use the extracted clinical data 130 to execute the detailed statistical analysis. In other words, the RWD can efficiently be used.

The present invention is not limited to the above embodiment and includes various modification examples. In addition, for example, the configurations of the above embodiment are described in detail so as to describe the present invention comprehensibly. The present invention is not necessarily limited to the embodiment that is provided with all of the configurations described. In addition, a part of each configuration of the embodiment may be removed, substituted, or added to other configurations.

A part or the entirety of each of the above configurations, functions, processing units, processing means, and the like may be realized by hardware, such as by designing integrated circuits therefor. In addition, the present invention can be realized by program codes of software that realizes the functions of the embodiment. In this case, a storage medium on which the program codes are recorded is provided to a computer, and a CPU that the computer is provided with reads the program codes stored on the storage medium. In this case, the program codes read from the storage medium realize the functions of the above embodiment, and the program codes and the storage medium storing the program codes constitute the present invention. Examples of such a storage medium used for supplying program codes include a flexible disk, a CD-ROM, a DVD-ROM, a hard disk, a solid state drive (SSD), an optical disc, a magneto-optical disc, a CD-R, a magnetic tape, a non-volatile memory card, and a ROM.

The program codes that realize the functions written in the present embodiment can be implemented by a wide range of programming and scripting languages such as assembler, C/C++, Perl, shell scripts, PHP, Python and Java.

It may also be possible that the program codes of the software that realizes the functions of the embodiment are stored on storing means such as a hard disk or a memory of the computer or on a storage medium such as a CD-RW or a CD-R by distributing the program codes through a network and that the CPU that the computer is provided with reads and executes the program codes stored on the storing means or on the storage medium.

In the above embodiment, only control lines and information lines that are considered as necessary for description are illustrated, and all the control lines and information lines of a product are not necessarily illustrated. All of the configurations of the embodiment may be connected to each other.

What is claimed is:

1. A computer system, comprising:
a processor;
a storage device coupled to the processor; and
a communication interface coupled to the processor,
wherein the computer system is coupled for access to a first database that stores clinical data including, as an item, information acquired on a clinical site and relating to a patient provided with a therapy and a second database that stores non-clinical data including, as an item, information acquired for an investigation purpose and relating to a patient group provided with a therapy,
wherein the processor is configured to:
select a predetermined number of pieces of the clinical data from the first database, and aggregate the selected predetermined number of pieces of the clinical data, to thereby generate comparative data; and
execute, through use of the non-clinical data and the comparative data which are the same in therapy content, first statistical analysis for analyzing a difference in the item, and record a result of the first statistical analysis.

2. The computer system according to claim 1,
wherein the clinical data and the non-clinical data include the item for identifying the therapy content, and
wherein the processor is configured to aggregate the selected predetermined number of pieces of the clinical data for each group of patients common in the therapy content, to thereby generate the comparative data.

3. The computer system according to claim 2,
wherein the result of the first statistical analysis includes identification information for identifying the therapy content, a type of the item, and a statistical index calculated in the first statistical analysis, and
wherein the processor is configured to:
receive an extraction condition including a conditional expression that uses a type of the item and a threshold value for the statistical index;
identify a result of the first statistical analysis that satisfies the extraction condition;
acquire, from the first database, the clinical data having target therapy content corresponding to the identification information included in the identified result of the first statistical analysis; and
execute, through use of the acquired clinical data and the non-clinical data having the target therapy content, second statistical analysis for analyzing the difference in the item, and record a result of the second statistical analysis.

4. The computer system according to claim 3, wherein the non-clinical data is data acquired from a paper and clinical study.

5. The computer system according to claim 3, wherein the therapy content is a combination of a plurality of therapies.

6. An information processing method, which is executed by a computer system,
the computer system including:
a processor;
a storage device coupled to the processor; and
a communication interface coupled to the processor,
the computer system being coupled for access to a first database that stores clinical data including, as an item, information acquired on a clinical site and relating to a patient provided with a therapy and a second database that stores non-clinical data including, as an item, information acquired for an investigation purpose and relating to a patient group provided with a therapy,
the information processing method including:
a first step of selecting, by the processor, a predetermined number of pieces of the clinical data from the first database, and aggregating the selected predetermined number of pieces of the clinical data, to thereby generate comparative data; and
a second step of executing, by the processor, through use of the non-clinical data and the comparative data which are the same in therapy content, first statistical analysis for analyzing a difference in the item, and recording a result of the first statistical analysis.

7. The information processing method according to claim 6,
wherein the clinical data and the non-clinical data include the item for identifying the therapy content, and
wherein the first step includes a step of aggregating, by the processor, the selected predetermined number of pieces of the clinical data for each group of patients common in the therapy content, to thereby generate the comparative data.

8. A non-transitory computer-readable storage medium storing a program for causing a computer to execute the following steps,
the computer including:
a processor;
a storage device coupled to the processor; and
a communication interface coupled to the processor,
the computer being coupled for access to a first database that stores clinical data including, as an item, information acquired on a clinical site and relating to a patient provided with a therapy and a second database that stores non-clinical data including, as an item, information acquired for an investigation purpose and relating to a patient group provided with a therapy,
the program causing the computer to execute:
a first step of selecting a predetermined number of pieces of the clinical data from the first database, and aggregating the selected predetermined number of pieces of the clinical data, to thereby generate comparative data; and
a second step of executing, through use of the non-clinical data and the comparative data which are the same in therapy content, first statistical analysis for analyzing a difference in the item, and recording a result of the first statistical analysis.

9. The non-transitory computer-readable storage medium according to claim 8,
wherein the clinical data and the non-clinical data include the item for identifying the therapy content,
wherein the first step includes a step of aggregating the selected predetermined number of pieces of the clinical data for each group of patients common in the therapy content, to thereby generate the comparative data, and
wherein the second step includes a step of executing the first statistical analysis through use of the non-clinical data and the comparative data which are the same in therapy content.
